(19) United States
(12) Patent Application Publication
(10) Pub. No.: US 2024/0325286 A1
(43) Pub. Date: Oct. 3, 2024

(54) HAIR GROWTH COMPOSITION AND METHODS THEREOF

(71) Applicant: Chelsea Ashira LLC, Marlton, NJ (US)

(72) Inventor: Chelsea Ashira SLOCOMBE, Marlton, NJ (US)

(21) Appl. No.: 18/619,650

(22) Filed: Mar. 28, 2024

Related U.S. Application Data
(60) Provisional application No. 63/455,008, filed on Mar. 28, 2023.

(51) Int. Cl. A61K 8/92; A61Q 5/00; A61Q 7/00

(52) U.S. Cl. CPC A61K 8/922; A61Q 5/002; A61Q 7/00

(57) ABSTRACT

A topical composition for stimulating hair follicles for hair regrowth, strengthening hair, thickening hair, moisturizing dry hair or scalp, or reducing dandruff or scalp irritation wherein the topical composition comprises an active component, which comprises one or more oils that nourish, strengthen, and/or moisturize the hair, roots, or scalp. The topical composition also comprises an essential oil component comprising one or more essential oils that provide a relieving or relaxing aroma to alleviate tension. The topical composition may be applied directly to the scalp, hair, or the area in which treatment is desired. Application can occur daily, weekly, fortnightly, or monthly. The topical composition may also be used in hot oil treatment of hair.

### RELATED APPLICATIONS AND INCORPORATION BY REFERENCE

This application claims priority under 35 U.S.C. § 119 (e) to U.S. Provisional Application No. 63/455,008, filed on Mar. 28, 2023, the entire contents of which are incorporated herein by reference.

### FIELD

The disclosed compositions and methods relate to a topical composition comprising natural oils and herbs to be applied to the human scalp to stimulate hair follicles for regrowth of hair, strengthening hair, thickening hair. The topical composition may also moisturize dry hair or the scalp or reduce dandruff or scalp irritation.

### BACKGROUND

Hair is produced within follicles, a small sac or cavity within the dermis. Within the follicle, cells divide and grow to build the hair shaft. Hair production occurs in three main phases: anagen phase, catagen phase, and telogen phase. The anagen phase is the active growth phase, where dividing cells grow the hair shaft, and for scalp hair, typically lasts several years. The catagen phase is a transition phase where, over a few weeks, the hair is cut off from the blood supply and dividing cells and the hair stops growing. The telogen phase is known as the resting phase, where the old hair detaches from the follicle. At any one time, hair on the human scalp is in one of these stages, although on normal scalps, most hairs are in the active growth phase, with 50-100 hairs shed daily. A blood supply delivers nutrients and hormones that regulate hair structure and growth, and sebaceous glands associated with the follicle produce sebum to help protect the skin and hair shaft.

The hair shaft is comprised of keratin, melanin (pigment), water, lipids and traces of minerals. The interior hair structure typically comprises an inner core known as the medulla (which may be absent) and a cortex, which gives hair its volume, texture and elasticity. The cortex contains melanin and fibrils of keratin embedded in a matrix with high water content. Inter- and intra-molecular interactions within the cortex, including pH dependent salt bonds, disulfide bonds, and hydrogen bonds between proteins and water, play a significant role in the elastic behavior of hair. A protective outer layer of dead keratinized cells, which form flat, smooth scales configured in an overlapping fashion, is referred to as the cuticle. Under normal conditions, the scales of the cuticle lie flat forming a protective barrier for the inner cortex and medulla, controlling water content of the hair fiber. Lipids act as an intracellular glue of the cortex and cuticle, and form an external film or layer over the cuticle, acting both as a protectant from damaging external factors and barrier to moisture loss. In this state, hair retains moisture, is protected from external influences, and is generally shiny and glossy in appearance, often being referred to as “healthy” hair.

However, numerous external factors, including environmental exposures, such as to UV radiation, wind, salt, chlorine, or pollution; mechanical forces, such as brushing, combing, pinning, twisting or binding the hair; the use of thermal appliances, including hair dryers, hot rollers and straightening irons; and chemical treatments, such as harsh shampoos, perming, coloring and relaxing; can disrupt the external lipid layer and cuticle. This can result in raised or even removed cuticle scales, exposing the cortex and making the hair vulnerable to water and lipid loss and keratin breakdown. Such damage renders the hair prone to tangling, frizz, and can cause tricoptilosis (also referred to as split ends)—the fraying of or stripping the cuticle from the hair shaft). In some instances, damage to the hair shaft can result in breakage, and even hair loss. The damaged hair can appear dull, is difficult to manage and style, often being described as “lifeless”.

Physiological factors, such as aging, hormonal changes, poor nutrition, physical and emotional stress, and certain diseases and conditions, as well as some medications and chemotherapies can also contribute negatively to the physical and chemical state of hair and the scalp, and adversely affect the hair's appearance, growth and loss.

For example, androgenic alopecia, is caused by the conversion of testosterone to dihydrotestosterone (DHT), which acts on hair follicles. This leads to hair follicle shrinkage with the hair becoming shorter and thinner with each cycle of growth, and is responsible for both male and female pattern baldness. In other examples, cases of severe stress can cause the hair to prematurely enter the telogen phase, resulting in noticeable hair loss. Certain medications may also cause hair loss.

Certain scalp conditions, such as scalp psoriasis, seborrheic dermatitis, and microbial infections can also have an adverse effect on hair. Scalp psoriasis is a common immune disorder that results in red, raised, often scaly, patches on the scalp. In mild cases there are only slight or moderate patches of scaling, however, in severe cases this can spread over the entire scalp and beyond the hairline, and manifest in thick crusted sores. Seborrheic dermatitis of the scalp may be characterized by flaky skin or crusty scales. Microbial infection, can lead to inflammation and a red, itchy scalp. While these conditions are of themselves not necessarily the direct cause of hair loss, intense itching and discomfort is often alleviated by scratching, which can, in severe cases, lead to hair loss.

Attempts to improve characteristics of hair, e.g. smoothness and shine, strength or elasticity, by treating or preventing damage caused by environmental, mechanical or chemical factors typically involve the application of agents, such as oils and synthetic agents, including water-soluble and non-soluble silicones, which act to provide a protective barrier around the cuticle and/or smooth and seal the damaged cuticle. Treatment for androgenic hair loss include anti-androgen medications that reduce androgen production or block the conversion of testosterone to DHT. Typical treatments for scalp conditions, such as scalp psoriasis and dermatitis, include topical treatments, for example, containing tar products, or salicylic acid, and include medicated shampoos, gels, creams and ointments. Topical or injectable steroids may also be used.

Many hair care products contain petroleum-derived and other highly manufactured ingredients. Such non-natural ingredients found in hair care products are potentially irritating, allergenic, antigenic, or toxic.

Furthermore, there is an increasing trend in consumer preferences for selecting natural and environmentally friendly products. According to publicly available information, the entire global hair care product market in 2019 was estimated at over $170 billion. The global market for natural hair care products has been growing and in 2019 was estimated at nearly $9 billion, with this trend expected to continue.

The awareness of the benefits of natural hair care products is one of the factors contributing to this market growth. With the preference by an increasing number of consumers for natural and environmentally friendly products, many of the more conventional products are seen as less desirable. There are many reasons for this preference towards natural products including environmental awareness and the avoidance of highly manufactured and refined products or products with artificial ingredients. Some consumers perceive detrimental effects with using more mainstream hair care products such as product harshness, scalp irritation, and poor hair quality such as dryness, split ends, and loss of sheen. Furthermore, individuals with product allergies or compromised immune systems, such as individuals undergoing chemotherapy, may not be able to use many of the mainstream commercial products. Consumers are also becoming more informed and savvier about product ingredients. These consumers carefully read product labels and avoid selecting products with ingredients such as sulfates, parabens, para-aminobenzoic acid, ethyl alcohol, mineral oil, and various additives and artificial ingredients.

Another area where there has been a need for innovative and natural hair care products is for wavy, curly, or coily hair, and in particular for the ethnic hair care market. There is a general relationship between ethnicity and hair types with Caucasian hair being the finest in diameter with the highest scalp density and variations in straightness or curliness, Asian hair being the coarsest in diameter with a medium scalp density and hair being the straightest, and African/Australoid hair being of variable diameter with the lowest scalp density and also generally of the curliest form. Based on these general hair type characteristics, there is consumer demand for natural hair care products that are particularly useful for treating and styling wavy, curly, or coily hair types.

Based on these trends and needs, consumers are also becoming increasingly aware of natural ingredients, such as plant-based ingredients or botanicals, and their suitability for a wide array of hair care products. Consequently, there is an increased interest in and demand for hair care products formulated with natural ingredients, and particularly for products designed for use with wavy, curly, or coily hair.

Not only women, but also male consumers are becoming increasingly aware of and using hair care products for their hair and beards. Many of these male consumers are also looking for naturally formulated products without petroleum-based and artificial ingredients.

Notwithstanding that there are cosmetic and pharmaceutical products on the market for hair and scalp treatments, many consumers can suffer sensitivities and unpleasant or harmful side effects, on contact with or exposure to some of the synthetic ingredients or medicaments used in hair and scalp treatments, or are concerned by potential adverse effects to these ingredients, and therefore seek treatments that reduce, minimize or avoid the use of any one or more such ingredients. In such instances, some consumers have a preference for products, which contain components that can be derived or are obtainable from botanic and other natural sources.

Citation or identification of any document in this application is not an admission that such document is available as prior art to the present application.

### SUMMARY

The disclosed formulations, compositions, and treatment methods relate to a topical composition for stimulating hair follicles for hair regrowth, strengthening hair, thickening hair, moisturizing dry hair or scalp, or reducing dandruff or scalp irritation.

In some embodiments, a topical composition comprises an active component and an essential oil component.

In some embodiments, an active component comprises at least one oil selected from the group consisting of camellia oil, safflower oil, jojoba oil, vitamin E oil, coconut oil, rose hip oil, moringa oil, grapeseed oil, marula oil, Jamaican black castor oil, olive oil, avocado oil, neem oil, brahmi infused sesame oil, and bhringraj infused sesame oil, and combinations thereof.

In some embodiments, an active component comprises about 5% to 15% camellia oil; about 10% to about 20% safflower oil; about 5% to about 15% Jojoba oil; about 0.01% to about 5% vitamin E oil; about 10% to about 25% coconut oil; about 0.01% to about 10% rose hip oil; about 0.01% to about 5% moringa oil; about 5% to about 15% grapeseed oil; about 0.01% to about 10% marula oil; about 0.01% to about 10% Jamaican black castor oil; about 0.01% to about 10% olive oil; about 5% to 15% avocado oil; about 0.01% to about 5% neem oil; about 0.01% to about 5% brahmi infused sesame oil; and about 0.01% to about 5% bhringraj infused sesame oil by volume of the composition.

In some embodiments, an essential oil component comprises at least one essential oil. In some embodiments, an at least one essential oil in the essential oil component is selected from the group consisting of peppermint oil, rosemary oil, tea tree oil, lemongrass oil, elemi oil, lavender oil, basil oil, eucalyptus oil, geranium oil, juniper oil, palmrose oil, passion fruit oil, patchouli oil, rosewood oil, sage oil, thyme oil, and ylang ylang oil, and combinations thereof.

In some embodiments, the essential oil component comprises about 0.01% to about 0.5% peppermint oil; about 0.01% to about 0.3% rosemary oil; about 0.01% to about 0.2% tea tree oil; about 0.01% to about 0.3% lemongrass oil; about 0.1% to about 0.2% elemi oil; about 0.01% to about 0.4% lavender oil; about 0.01% to about 0.1% basil oil; about 0.01% to about 0.1% eucalyptus oil; about 0.01% to about 0.1% geranium oil; about 0.01% to about 0.1% juniper oil; about 0.01% to about 0.1% palmrose oil; about 0.01% to about 0.1% passion fruit oil; about 0.01% to about 0.1% patchouli oil; about 0.01% to about 0.1% rosewood oil; about 0.01% to about 0.1% sage oil; about 0.01% to about 0.1% thyme oil; and about 0.01% to about 0.1% ylang ylang oil by volume of the composition.

In some embodiments, a topical composition comprises an active component comprising (in v/v %): about 5% to 15% camellia oil; about 10% to about 20% safflower oil; about 5% to about 15% Jojoba oil; about 0.01% to about 5% vitamin E oil; about 10% to about 25% coconut oil; about 0.01% to about 10% rose hip oil; about 0.01% to about 5% moringa oil; about 5% to about 15% grapeseed oil; about 0.01% to about 10% marula oil; —about 0.01% to about 10% Jamaican black castor oil; about 0.01% to about 10% olive oil; about 5% to 15% avocado oil; about 0.01% to about 5% neem oil; about 0.01% to about 5% brahmi infused sesame oil; and about 0.01% to about 5% bhringraj infused sesame oil, and the essential oil component comprises: about 0.01% to about 0.5% peppermint oil; about 0.01% to about 0.3% rosemary oil; about 0.01% to about 0.2% tea tree oil; about 0.01% to about 0.3% lemongrass oil; about 0.1% to about 0.2% elemi oil; about 0.01% to about 0.4% lavender oil; about 0.01% to about 0.1% basil oil; about 0.01% to about 0.1% eucalyptus oil; about 0.01% to about 0.1% geranium oil; about 0.01% to about 0.1% juniper oil; about 0.01% to about 0.1% palmrose oil; about 0.01% to about 0.1% passion fruit oil; about 0.01% to about 0.1% patchouli oil; about 0.01% to about 0.1% rosewood oil; about 0.01% to about 0.1% sage oil; about 0.01% to about 0.1% thyme oil; and about 0.01% to about 0.1% ylang ylang oil.

In some embodiments, the active component is at least 97% by volume of the composition.

In some embodiments, the active component is at least 98.5% by volume of the composition.

In some embodiments, the essential oil component is at most 3% by volume of the composition.

In some embodiments, a volume ratio of coconut oil to safflower oil ranges from 0.5:1 to 2.5:1.

In some embodiments, the volume ratio of coconut oil to safflower oil is 1.33:1.

A topical composition may include natural oils and herbs in the active and essential oil components. Thus, the topical composition does not contain silicones, sulfates, petroleums, mineral oil, parabens, phthalates, and formaldehyde. Despite comprising natural oils and herbs, in some embodiments, the topical composition does not contain any nut-based oils.

In some embodiments, the topical composition comprises an active component, wherein the active component comprises: 8 fl. oz. camellia oil; 12 fl. oz. safflower oil; 6 fl. oz. Jojoba oil; 2 fl. oz. vitamin E oil; 16 fl. oz. coconut oil; 4 fl. oz. rose hip oil; 2 fl. oz. moringa oil; 8 fl. oz. grapeseed oil; 4 fl. oz. marula oil; 4 fl. oz. Jamaican black castor oil; 4 fl. oz. olive oil; 8 fl. oz. avocado oil; 2 fl. oz. neem oil; 2 fl. oz. brahmi infused sesame oil; and 2 fl. oz. bhringraj infused sesame oil, and an essential oil component, wherein the essential oil component comprises: 1.25 tsp peppermint oil; 0.75 tsp rosemary oil; 0.5 tsp tea tree oil; 0.75 tsp lemongrass oil; 0.5 tsp elemi oil; 1 tsp lavender oil; 0.25 tsp basil oil; 0.25 tsp eucalyptus oil; 0.25 tsp geranium oil; 0.25 tsp juniper oil; 0.25 tsp palmrose oil; 0.25 tsp passion fruit oil; 0.25 tsp patchouli oil; 0.25 tsp rosewood oil; 0.25 tsp sage oil; 0.25 tsp thyme oil; and 0.25 tsp ylang ylang oil.

Methods of stimulating hair follicles for hair regrowth, strengthening hair, thickening hair, moisturizing dry hair or scalp, or reducing dandruff or scalp irritation are also disclosed herein.

In some embodiments, a method for stimulating hair follicles for hair regrowth, strengthening hair, thickening hair, moisturizing dry hair or scalp, or reducing dandruff or scalp irritation comprising applying a topical composition to a scalp, the composition comprising: an active component comprising at least one oil selected from the group consisting of camellia oil, safflower oil, jojoba oil, vitamin E oil, coconut oil, rose hip oil, moringa oil, grapeseed oil, marula oil, Jamaican black castor oil, olive oil, avocado oil, neem oil, brahmi infused sesame oil, and bhringraj infused sesame oil, and combinations thereof, and an essential oil component comprising at least one essential oil selected from the group consisting of peppermint oil, rosemary oil, tea tree oil, lemongrass oil, elemi oil, lavender oil, basil oil, eucalyptus oil, geranium oil, juniper oil, palmrose oil, passion fruit oil, patchouli oil, rosewood oil, sage oil, thyme oil, and ylang ylang oil, and combinations thereof.

In some embodiments, applying may include applying between about 0.5 mL to about 30 mL of the composition to the scalp. The applying may comprise application of the entire volume of the composition used for treatment at once or the applying may comprise application of the volume for treatment gradually and in small parts during the course of the treatment, for example, applying the composition dropwise using a dropper, or dividing the treatment volume into smaller portions (e.g. 3×5 mL portions for a 15 mL treatment volume) such that the total volume is applied in multiple, smaller volumes as application of an excessive volume can lead to a volume of the composition that is lost as runoff (e.g. dripping down off of the scalp down the neck, dripping off of the scalp or hair onto another surface).

In some embodiments, the applying may occur daily. In some embodiments, the applying may occur every other day. In some embodiments, the applying may occur weekly. In some embodiments, the applying may occur biweekly.

The present topical composition can be used for sealing in moisture in the hair. In some embodiments, applying the topical composition to the scalp occurs before applying a hair cream. In some embodiments, the step of applying the topical composition to the scalp occurs after applying a hair cream.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited.

These and other embodiments are disclosed or are obvious from and encompassed by, the following Detailed Description.

### BRIEF DESCRIPTION OF THE DRAWINGS

The following detailed description, given by way of example, but not intended to limit the formulations, compositions, and methods to the specific embodiments described, may best be understood in conjunction with the accompanying drawings.

FIG. 1 is a photograph showing a female subject's hair before application of the topical composition and after application of the topical composition following a treatment course of five months.

FIG. 2 is a photograph showing a female subject's hair before application of the topical composition, after one week of daily application of the topical composition, and two weeks after daily application of the topical composition.

FIG. 3 is a photograph showing a female subject's hair before application of the topical composition and after application of the topical composition following a treatment course.

FIG. 4 is a photograph showing a female subject's hair before application of the topical composition, after one week of daily application of the topical composition, and four weeks after daily application of the topical composition.

FIG. 5 is a photograph showing a female subject's hair before application of the topical composition, after one week of daily application of the topical composition, and four weeks after daily application of the topical composition.

FIG. 6 is a photograph showing a subject's hair, before application of the topical composition and four weeks after daily application of the topical composition wherein the subject has noticeable bald spots between braids prior to application of the topical composition. These bald spots exhibited hair growth during the four-week course of treatment comprising daily application of the topical composition.

FIG. 7 is a photograph showing a male subject's hair before application of the topical composition, after five days of daily application of the topical composition, and four weeks after daily application of the topical composition.

FIG. 8 is a photograph showing a female subject's hair, exhibiting female pattern baldness, before application of the topical composition, after nine days of daily application of the topical composition, and two weeks after daily application of the topical composition.

FIG. 9 is a photograph showing a male subject's hair having short dreadlocks prior to application of the topical composition, and after one year of application 2-3 times weekly to stimulate hair growth, the hair contained within the dreadlocks was observed to grow both longer and thicker.

FIG. 10 is a photograph showing a female subject having short hair before application of the topical composition, and after one year of application of the topical composition 2-3 times weekly to stimulate hair growth, the subject exhibited an increased hair growth rate, resulting in increased hair length.

### DETAILED DESCRIPTION

The present topical compositions do more than stimulate hair growth. They also may moisturizes and conditions the user's hair, controls dandruff and itchy scalp, treats psoriasis, repairs damaged hair, controls frizzy hair, gives hair a smooth look and feel, minimizes hair shedding, strengthens and thickens the hair, makes hair softer and more manageable, builds hair body, and restores luster and shine to the hair. The topical compositions are envisioned to be used on all types of hair. In some embodiments, a topical composition is an oil-like composition.

In some embodiments, the topical composition can comprise an active component and an essential oil component. The active component comprises one or more oils that has the ability to stimulate hair growth, strengthen the hair, moistens the hair, and moisten and treat the scalp to minimize itchiness and the development of dandruff.

In some embodiments, the active component comprises a combination of oils. The oils can be synthetic or from natural sources. Preferably, the oils are free of synthetic components such as silicones, sulfates, petroleums, mineral oil, parabens, phthalates, and formaldehyde. Even more preferably, the oils in the active component are from natural sources. Examples of oils that can be used in the active component include, but are not limited to camellia oil, safflower oil, jojoba oil, vitamin E oil, coconut oil, rose hip oil, moringa oil, grapeseed oil, marula oil, Jamaican black castor oil, olive oil, avocado oil, neem oil, brahmi infused sesame oil, and bhringraj infused sesame oil, and combinations thereof.

Camellia oil is derived from flowering plants in the family Theaceae and typically found in eastern and southern Asia. Camellia oil is commonly known as tea oil. The oil obtained from pressing the seeds of C. japonica is used in hair care and can be called tsubaki oil or tsubaki-abura. Camellia oil contains proteins, which fortify hair strands, glycerides, which help prevent the loss of moisture, and vitamin E, which moisturizes the hair.

Safflower oil is derived from seeds of safflower and is commonly used as a component of vegetable oil in cooking applications. Safflower oil primarily comprises a mixture of fatty acids and includes oleic, linoleic, palmitic, stearic, linolenic, and palmitoleic acids.

Jojoba oil is produced in the seed of Simmondsia chinensis. Although having the appearance of an oil, jojoba oil is a wax ester, comprising fatty acid esters.

Vitamin E oil comprises one or a mixture of four tocopherols or tocotrienols. Vitamin E is an antioxidant, which helps to prevent oxidation and the formation of free radicals, a reactive oxygen species. An imbalance of reactive oxygen species can lead to damage from oxidative stress. Vitamin E acts to protect cell membranes from oxidation by reacting with lipid radicals. With respect to hair care, vitamin E prevents healthy hair follicle cells from breaking down, which could lead to thinning, hair breakage, and/or hair loss.

Coconut oil is derived from the wick, meat, and milk of coconuts. Coconut oil is about 99% fat and primarily comprises saturated fat (˜82%). Most of the saturated fat in coconut oil is lauric acid. Other significant saturated fats found in coconut oil include myristic acid, palmitic acid, and caprylic acid. Monounsaturated fats and polyunsaturated fats make up a small percentage of the fat content of coconut oil. Coconut oil helps to reduce the amount of hair protein (keratin) that is lost when other hair care or styling products are applied to the hair. The damage to keratin renders the hair dull and fragile. Coconut oil has also been shown to help reduce or eliminate dandruff.

Rosehip oil is derived from the seeds and fruit of the Rosa canina bush. Rosehip oil is rich in fatty acids such as gamma-linolenic oil, which serves as a conditioner to nourish and soften hair, as well as a natural moisturizer to prevent the scalp from drying out, reducing or eliminating dandruff.

Moringa oil is produced from the seeds of moringa oleifera. Moringa oil comprises about 40% monosaturated fatty acids such as oleic acid, whose benefits include maintenance of the softness, suppleness, and radiance of skin and hair, stimulation of the growth of thicker, longer, and stronger hair, reduction in the appearance of aging, such as premature wrinkles and fine lines, elimination of dandruff, boosting immunity, prevention of joint inflammation, stiffness, and pain, and antioxidant properties.

Grapeseed oil is derived from the seeds of grapes. The fatty acids commonly found in grapeseed oil include linoleic acid, oleic acid, palmitic acid, stearic acid, alpha-linolenic acid, and palmitoleic acid. Grapeseed oil can help to moisten, strengthen, and shine hair and can also help moisturize the scalp to loosen dead skin and retain moisture, reducing or eliminating dandruff. Marula oil is derived from the seeds and nuts of Marula trees (Sclerocarya birrea). The fatty acids commonly found in marula oil include oleic acid, linoleic acid, alpha-linoleic acid, palmitic acid, stearic acid, and arachidonic acid. Marula oil is a moisturizer that can be used to strengthen hair, enhance its shine, and fight frizz. Marula oil also contains antioxidants such as vitamin E and vitamin C that can help to repair damaged hair and prevent split ends from forming.

Jamaican black castor oil is derived from castor beans, which are typically roasted before grinding and boiled for extraction of the oil. This gives the oil a distinct dark brown color and burnt smell. Among other fatty acids, Jamaican black castor oil contains ricinoleic acid, which may help stimulate hair growth as well as increase the absorption of nutrients into the hair and scalp.

Olive oil is derived from the fruit of olive trees and is commonly used in cooking and food preparation. Olive oil primarily comprises the monounsaturated fatty acid, oleic acid, in addition to linoleic acid, palmitic acid, stearic acid, and alpha-linolenic acid. Olive oil can help in retaining moisture in the hair, which helps to reduce damage, shrinkage, and frizz as the monounsaturated fatty acid can easily penetrate the hair follicle.

Avocado oil is derived from the pulp of avocados. Like olive oil, avocado oil is high in monounsaturated fat content, in particular, oleic acid. Other fatty acids present in olive oil include palmitic acid, linoleic acid, docosadienoic acid, palmitoleic acid, linolenic acid, eicosenoic acid, and myristic acid. The high content of monounsaturated fatty acid is able to penetrate the hair follicle to help retain moisture and reduce damage and frizz. Avocado oil also helps to moisturize and soothe the scalp, reducing dryness and flakiness, reducing or eliminating dandruff.

Neem oil, also known as margosa oil, is derived from the fruits and seeds of the neem tree. Neem oil also is a mixture of fatty acids such as oleic, stearic, palmitic, linoleic, arachidic, and myristic acids. The fatty acid composition helps to moisturize the scalp to nourish hair stands and control frizz. Neem oil also contains triglycerides, steroids (campesterol, beta-sitosterol, stigmasterol) and many triterpenoids such as nimbin and azadirachtin. Neem oil can also have anti-inflammatory, antipyretic, fungicidal, antihistamine and antisep tic properties, inhibiting growth of bacteria that can cause dandruff, folliculitis, and hair loss. Neem oil also has insecticidal properties and may be used in head lice treatment.

Brahmi is an herb (Bacopa monneiri) whose oil may be used to prevent split ends through decreasing dryness and sealing in moisture in hair follicles. This gives the hair a smooth texture and shine. Brahmi is also rich in antioxidants such as vitamin C, saponins, and falvonoids, which may strengthen the roots to decrease hair loss. In some embodiments, the brahmi in the composition comprises a brahmi oil base. In some embodiments, the brahmi may be infused with another oil as its base. In some embodiments, the brahmi is infused in sesame oil.

Bhringraj is a plant (Eclipta prostrata) that may be used in the treatment and prevention of baldness. Bhringraj helps to increase blood circulation in the scale and hair follicles to permit more nutrients to flow via the blood supply. This enriches hair roots and promotes hair growth. In some embodiments, the bhringraj in the composition comprises a bhringraj oil base. In some embodiments, the bhringraj may be infused with another oil as its base. In some embodiments, the bhringraj is infused in sesame oil.

As previously described, many of the oils of the active component comprise mixtures of fatty acids, which include monounsaturated fatty acids and polyunsaturated fatty acids.

The benefits of oleic acid, also known as omega-9 fatty acid, include maintenance of the softness, suppleness, and radiance of skin and hair, stimulation of the growth of thicker, longer, and stronger hair, reduction in the appearance of aging, such as premature wrinkles and fine lines, elimination of dandruff, boosting immunity, prevention of joint inflammation, stiffness, and pain, and antioxidant properties.

The benefits of linoleic acid, also known as omega-6 fatty acid, include moisturizing hair and promoting its growth, facilitating wound healing, anti-inflammatory properties, promoting moisture retention in skin and the hair, and slowing the appearance of aging by sustaining skin elasticity and softness.

Palmitic acid is a common emollient and its benefits include the softening of hair and the skin.

Stearic acid has cleansing properties that eliminate dirt, sweat, and excess sebum from hair and skin. Stearic acid also conditions and protects hair from damage without diminishing luster or making it feel heavy.

The benefits of linolenic acid, also known as omega-3 fatty acid, include the lessening of inflammation, the control of blood clotting on the skin, and the ability to soothe joint pain and case stiffness to improve flexibility. With respect to hair care, linolenic acid combats dryness of the hair and can stimulate hair follicles to grow. Furthermore, linolenic acid helps to inhibit the activity of 5α-reductase, an enzyme whose activity contributes to hair loss.

The benefits of palmitoleic acid include delaying the appearance of premature aging, moisturizing and tightening the skin, promoting the growth of shining hair, boosting the growth of healthy-looking nails, and enhancing skin elasticity to prevent symptoms of premature aging, such as wrinkles.

Essential oils are concentrated hydrophobic liquids containing volatile chemical compounds from plants. The term “essential” indicates that the oil contains the essence of the plant from which the oil was extracted and does not relate to indispensability. Essential oils are plant extracts and are obtained by steaming and/or pressing the various parts of a plant to capture the compounds.

Essential oils are typically used in aromatherapy, which involves the use of aromatic materials for the improvement of psychological and physical well-being. The aromas may also induce calming and relaxation. Thus, the essential oil component of the present topical composition relates to stimulating the olfactory nerves through the aroma of the oils, which impact the amygdala, the emotional center of the brain. Essential oils can also can also be absorbed by the skin and can help alleviate tension and soreness in the muscles.

Any essential oil can be used in the essential oil component of the present topical composition. Examples of essential oils that can be used include, but are not limited to peppermint oil, rosemary oil, tea tree oil, lemongrass oil, elemi oil, lavender oil, basil oil, eucalyptus oil, geranium oil, juniper oil, palmrose oil, passion fruit oil, patchouli oil, rosewood oil, sage oil, thyme oil, and ylang ylang oil, and combinations thereof.

In some embodiments, the active component comprises from about 80% to about 99% by volume, or from about 85% to about 98% by volume, or from about 88% to about 96% by volume, or from about 90% to about 95% by volume, or about 90% by volume, or about 91% by volume, or about 92% by volume, or about 93% by volume, or about 94% by volume, or about 95% by volume, or about 96% by volume, or about 97%, or about 98% by volume, or about 99% by volume of the composition.

In some embodiments, the active component comprises at least 97% by volume of the composition. In some embodiments, the active component comprises at least 98.5% by volume of the composition.

Each oil in the active component can be present in any amount and the dominant oil or oils in the active component, which is present in plurality compared to the other oils in the active component, may be the same or different and the dominant oil or oils may change depending on the desired application. In some embodiments the oils in the active component may be present in an amount from about 0.5% to about 98.5%, or from about 1% to about 98%, or from about 2% to about 97%, or from about 3% to about 96%, or from about 4% to about 95%, or from about 5% to about 94%, or from about 6% to about 93%, or from about 7% to about 92%, or from about 8% to about 91%, or from about 9% to about 90%, or from about 10% to about 89%, or from about 11% to about 88%, or from about 12% to about 87%, or from about 13% to about 86%, or from about 14% to about 85%, or from about 15% to about 84%, or from about 16% to about 83%, or from about 17% to about 82%, or from about 18% to about 81%, or from about 19% to about 80%, or from about 20% to about 79%, or from about 25% to about 74%, or from about 30% to about 69%, or from about 35% to about 64%, or from about 40% to about 59%, or from about 45% to about 54%, or about 50% by volume of the composition.

In some embodiments, safflower oil and coconut oil predominate the active component. A volume ratio between these two predominant oils in the active component can be ascertained and varied depending on the desired application. The volume ratio of coconut oil to safflower oil can range from about 0.5:1 to about 10:1, or from about 1:1 to about 9:1, or from about 1.5:1 to about 8:1, or from about 2:1 to about 7:1, or from about 2.5:1 to about 6:1, or about 3:1 to about 5:1, or about 4:1. In some embodiments, the volume ratio of coconut oil to safflower oil ranges from about 0.5:1 to about 2.5:1. In some embodiments, the volume ratio of coconut oil to safflower oil is about 1.33:1.

In some embodiments, the essential oil component comprises from about 20% to about 1% by volume, or from about 15% to about 2% by volume, or from about 12% to about 4% by volume, or from about 10% to about 5% by volume, or about 10% by volume, or about 9% by volume, or about 8% by volume, or about 7% by volume, or about 6% by volume, or about 5% by volume, or about 3% by volume, or about 2% by volume, or about 1% by volume of the composition.

In some embodiments, the essential oil component comprises no more than 3% by volume of the composition. In some embodiments, the essential oil component comprises no more than 1.5% by volume of the composition.

Because the present topical composition comprises mostly oils in the active component and essential oil component, in some embodiments, the topical composition is substantially free of water. In some embodiments, the topical composition contains less than about 10% by volume of water, or less than about 5% by volume of water, or less than about 2.5% by volume of water, or less than about 2% by volume of water, or less than about 1.5% by volume of water, or less than about 1% by volume of water, or less than about 0.5% of by volume water, or less than about 0.25% by volume of water, or less than about 0.1% by volume of water. The water content can ideally be 0% by volume of water or as practically close to 0% such that no appreciable amount of water is intended to be added or incorporated into the composition, such that the composition is substantially free of water.

The present topical composition can be made using standard formulation and mixing techniques familiar to one of ordinary skill in the art of formulations.

In some embodiments, the oils of the active component and the essential oil component are combined with mixing to achieve a uniform, homogeneous mixture. Any equipment may be used to contact the mixture as long as uniform distribution of the mixture liquid is achieved. For example, small-scale production can be achieved by mixing in jars or stainless steel bowls, while for larger quantities, planetary mixers may be used.

Mixing may be accomplished by blending, stirring, shaking, tumbling, rolling, or by any other method to achieve a uniform, homogeneous blend. In some embodiments, the components to be mixed are combined under low shear conditions in a suitable apparatus capable of functioning under low shear conditions.

The mixing time can be any time that is needed to achieve the uniform, homogeneous blend and varies by the requisite production scale. For small scale production, the mixing time ranges from about 1 minute to about 60 minutes, or from about 5 minutes to about 55 minutes, or from about 10 minutes to about 50 minutes, or from about 15 minutes to about 45 minutes, or from about 20 minutes to about 40 minutes, or about 30 minutes.

The mixture can be optionally heated while mixing. The temperature range of heating can range from about 100° F. to about 120° F., or about 110° F. to about 130° F., or about 120° F. to about 140° F., or about 130° F. to about 150° F., or about 150° F. to about 200° F.

The present topical composition is not limited to a liquid topical and can be made into lotions, creams, gels, sticks, sprays, ointments, cleansing liquid washes, solid bars, shampoos, pastes, powders, foams, mousses, and wipes.

The topical composition may be utilized by topical application of said composition to the area of the body on which treatment is desired. Treatment may include, but is not limited to stimulating hair follicles for hair regrowth, strengthening hair, thickening hair, moisturizing dry hair or scalp, or reducing dandruff or scalp irritation.

The topical composition may be applied directly to the hair or scalp by any suitable means, including, by hand, with a spatula, scoop or spoon, spraying, dusting, painting and pouring. The composition is preferably massaged into the hair and/or scalp. While some oils, such as rose hip, jojoba, olive, and coconut oils are rapidly absorbed into the hair (about 2-4 hours), others, such as avocado and Jamaican black castor oils are absorbed more slowly, (about 6-8 hours). Thus, while some benefit may be obtained while the oil-based compositions are left on for 2-4 hours, oil-based compositions are preferably left on for at least about 6-8 hours, or longer, such as 8-12 hours, and may conveniently be left on overnight. Preferably, the topical composition remains in the hair and scalp following application and is not washed or rinsed off.

The topical composition may also be used as a sealant to lock in and retain moisture in the hair following use of other hair care products such as creams. The topical composition is preferably applied to the hair or scalp following treatment or application of other hair care products. Preferably, the topical composition remains in the hair and scalp following application and is not washed or rinsed off.

The topical composition may also be applied to the hair or scalp as part of a hot oil treatment. Typically, the topical composition is heated by any means such as stovetop or a microwave and applied to the scalp and hair. After letting the composition absorb into the scalp and hair, the composition may be washed or rinsed off. In hot oil treatment, the topical composition is heated to a temperature between about 75 to about 150° F., or about 100° F. to about 125° F., or about 125° F.

A treatment process may be performed as a single, one-off treatment, or may be used on a regular basis over a period of time, for example, the composition may be applied daily, once or twice weekly, fortnightly, or monthly.

Any amount of the topical composition may be applied to the hair or scalp during treatment. The amount to apply depends on the size of the area to be treated and the relative volume of hair of the user. About 0.5 to about 30 mL, or about 1 to about 29 mL, or about 5 to about 25 mL, or about 10 to about 20 mL, or about 15 mL of the topical composition may be applied to the hair or scalp. In some embodiments, about 50 mL of the topical composition is applied to the hair or scalp.

The topical composition may be used on any and all types of hair, which include, but are not limited to wavy, curly, or coily hair. The hair may be of its natural color (e.g. black, brown, blonde, red, auburn) or it may be gray as a result of natural aging or other physiological condition. The hair may or may not have color treatments such as highlights or dyes that result in an unnatural hair color (e.g. blue, pink, green, purple, bright red, or combinations thereof) and application of the topical composition does not affect the unnatural hair color. The topical composition may also be used on any and all scalp types, which include, but are not limited to balanced, oily, or dry scalps.

The average hair density of a human scalp ranges from about 100-200 hairs per square inch of the scalp. Those with higher hair densities may have a hair density of 300-400 hairs per square inch and those with lower hair densities may have about 50-100 hairs per square inch. Areas of baldness have even lower hair density, even going as low as 0 hairs per square inch. Thus, thickening the hair increases hair density in the user. Thus, in users with lower hair density, application of the topical composition increases the hair density of the user. In some embodiments, application of the topical composition, increases the hair density by about 10 hairs per square inch of a scalp, by about 15 hairs per square inch of the scalp, about 20 hairs per square inch of the scalp, about 25 hairs per square inch of the scalp, about 30 hairs per square inch of the scalp, about 35 hairs per square inch of the scalp, about 40 hairs per square inch of the scalp, about 45 hairs per square inch of the scalp, about 50 hairs per square inch of the scalp, about 75 hairs per square inch of the scalp, about 100 hairs per square inch of the scalp, about 125 hairs per square inch of the scalp, about 150 hairs per square inch of the scalp, about 175 hairs per square inch of the scalp, about 200 hairs per square inch of the scalp, about 250 hairs per square inch of the scalp, or about 300 hairs per square inch of the scalp. In some embodiments, application of the topical composition to a bald spot results in the growth of hair and increase in hair density at the bald spot by about 10 hairs per square inch of a scalp, by about 15 hairs per square inch of the scalp, about 20 hairs per square inch of the scalp, about 25 hairs per square inch of the scalp, about 30 hairs per square inch of the scalp, about 35 hairs per square inch of the scalp, about 40 hairs per square inch of the scalp, about 45 hairs per square inch of the scalp, about 50 hairs per square inch of the scalp, about 75 hairs per square inch of the scalp, about 100 hairs per square inch of the scalp, about 125 hairs per square inch of the scalp, about 150 hairs per square inch of the scalp, about 175 hairs per square inch of the scalp, about 200 hairs per square inch of the scalp, about 250 hairs per square inch of the scalp, or about 300 hairs per square inch of the scalp.

The present topical composition can also stimulate the rate at which hair grows and permits users to speed up the rate of hair growth to achieve longer hair lengths in shorter amounts of time. Typically, human hair grows at about 0.35 mm per day and about 0.5 cm to about 1.7 cm in one month. In some embodiments, application of the topical composition increases the rate at which hair grows per month by about 0.1 cm per month, by about 0.2 cm per month, by about 0.3 cm per month, by about 0.4 cm per month, by about 0.5 cm per month, by about 0.6 cm per month, or by about 0.7 cm per month.

Although the formulations, compositions, and methods have been described in detail, it should be understood that various changes, substitutions and alterations can be made herein without departing from the spirit and scope of the invention as defined in the appended claims.

The following Examples which are given for illustration purposes only and are not intended to limit the invention in any way.

### EXAMPLES

### Example 1

A typical composition comprises the following components:

| | | | Volume | Percent volume |
|---|---|---|---|---|
| Component | Amount | | (mL) | (% v/v) |
| Camellia oil | 8 | fl. oz. | 236.6 | 9.38 |
| Safflower oil | 12 | fl. oz. | 354.9 | 14.08 |
| Jojoba oil | 6 | fl. oz. | 177.4 | 7.04 |
| Vitamin E oil | 2 | fl. oz. | 59.1 | 2.35 |
| Coconut oil | 16 | fl. oz. | 473.2 | 18.77 |
| Rose hip oil | 4 | fl. oz. | 118.3 | 4.69 |
| Moringa oil | 2 | fl. oz. | 59.1 | 2.35 |
| Grapeseed oil | 8 | fl. oz. | 236.6 | 9.38 |
| Marula oil | 4 | fl. oz. | 118.3 | 4.69 |
| Jamaican black | 4 | fl. oz. | 118.3 | 4.69 |
| castor oil | | | | |
| Olive oil | 4 | fl. oz. | 118.3 | 4.69 |
| Avocado oil | 8 | fl. oz. | 236.6 | 9.38 |
| Neem oil | 2 | fl. oz. | 59.1 | 2.35 |
| Brahmi infused | 2 | fl. oz. | 59.1 | 2.35 |
| sesame oil | | | | |
| Bhringraj infused | 2 | fl. oz. | 59.1 | 2.35 |
| sesame oil | | | | |
| Peppermint oil | 1.25 | tsp | 6.2 | 0.24 |
| Rosemary oil | 0.75 | tsp | 3.7 | 0.15 |
| Tea tree oil | 0.5 | tsp | 2.5 | 0.10 |
| Lemongrass oil | 0.75 | tsp | 3.7 | 0.15 |
| Elemi oil | 0.5 | tsp | 2.5 | 0.10 |
| Lavender oil | 1.0 | tsp | 4.9 | 0.20 |
| Basil oil | 0.25 | tsp | 1.2 | 0.05 |
| Eucalyptus oil | 0.25 | tsp | 1.2 | 0.05 |
| Geranium oil | 0.25 | tsp | 1.2 | 0.05 |
| Juniper oil | 0.25 | tsp | 1.2 | 0.05 |
| Palmrose oil | 0.25 | tsp | 1.2 | 0.05 |
| Passion fruit oil | 0.25 | tsp | 1.2 | 0.05 |
| Patchouli oil | 0.25 | tsp | 1.2 | 0.05 |
| Rosewood oil | 0.25 | tsp | 1.2 | 0.05 |
| Sage oil | 0.25 | tsp | 1.2 | 0.05 |
| Thyme oil | 0.25 | tsp | 1.2 | 0.05 |
| Ylang ylang oil | 0.25 | tsp | 1.2 | 0.05 |
| | | | 2521.2 mL total | 100% |

The above table displays the relative amounts of the active component (in volume units fl. oz.) and the essential oil component (in volume units tsp) in an exemplary topical composition. Fluid ounces (fl. oz.) were converted to milliliters (mL) using a conversion factor of 29.574 mL per fl. oz. Teaspoons (tsp) were converted to milliliters (mL) using a conversion factor of 4.929 mL per tsp.

The composition was prepared by combining each component at room temperature and mixed to obtain a homogeneous composition. Any form of mixing can be used to stir and mix the components of the composition, as long as the components are even blended and the mixture is a homogenous solution. General methods of stirring and mixing can include, but are not limited to mixing by hand, with or without the assistance of a tool or utensil such as a mixing spoon, stand mixers, or industrial mixers.

### Example 2

A typical application regimen is as follows. 1 tablespoon of the topical composition (˜15 mL) is applied to the scalp and massaged for at least one minute to allow the scalp to absorb the composition. The composition may be applied to the scalp all at once or dropwise, for example, using a dropper. The composition is left in the hair and is not rinsed out. The regimen is repeated daily.

The amount of topical composition used should be sufficient to cover the area of the scalp and the overall amount may vary depending on the area needed to cover and the volume of the subject's hair as the composition could easily become trapped in thick hair and not be absorbed by the roots.

### Example 3

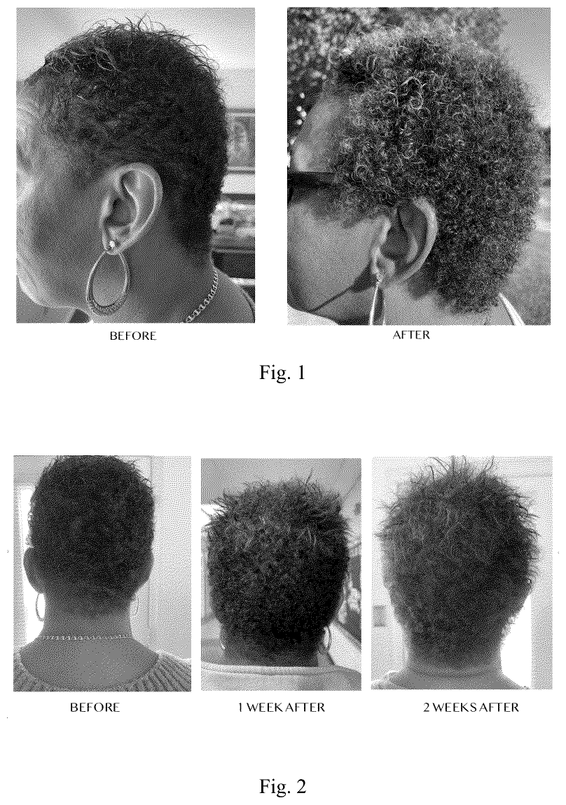

In referring to FIG. 1, it can be seen that the subject's hair prior to treatment is thin. Daily application of the topical composition for as described in Example 2 stimulated hair growth and resulted in thicker and more voluminous hair over a treatment course of five months.

### Example 4

In referring to FIG. 2, it can be seen that the subject's hair prior to treatment is thin. The hair was noticeably thicker after just one week of application of the topical composition. Daily application of the topical composition over the course of two weeks as described in Example 2 stimulated hair growth and resulted in thicker and more voluminous hair as observed one week and two weeks following beginning the course of treatment.

### Example 5

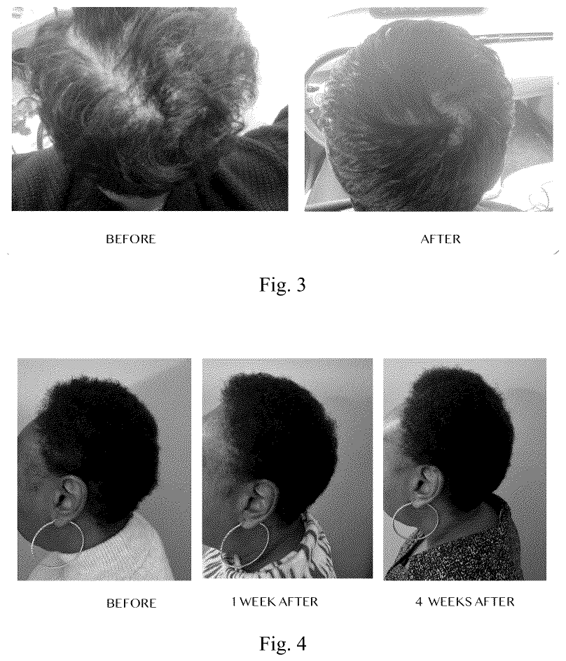

In referring to FIG. 3, it can be seen that the subject, prior to treatment, exhibits a bald patch along the hairline. Daily application of the topical composition for four weeks as described in Example 2 stimulated hair growth and resulted in hair growth at the bald patch, which was sufficient to cover up the bald patch.

### Example 6

In referring to FIG. 4, the subject has thinner hair prior to treatment. The hair was noticeably thicker after just one week of application of the topical composition. Daily application of the topical composition for four weeks as described in Example 2 stimulated hair growth and resulted in hair growth in which the hair is noticeably thicker and more voluminous.

### Example 7

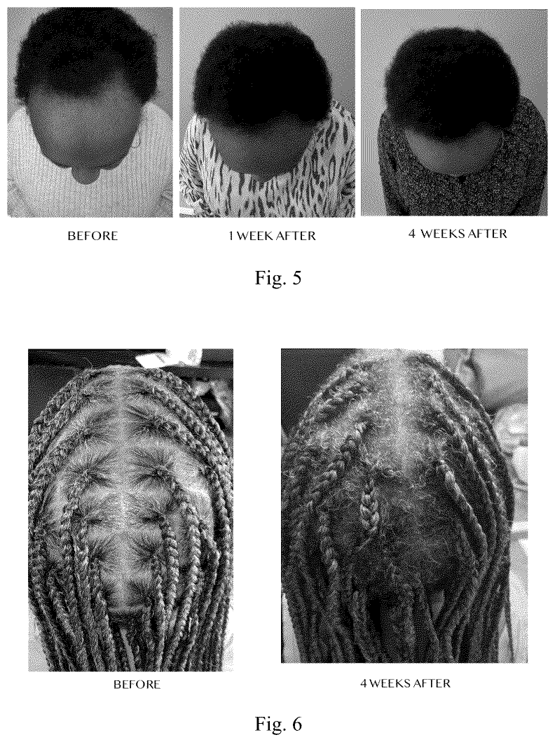

In referring to FIG. 5, the subject prior to treatment has a bald spot on the left hand side of the forehead. Hair began to grow at the bald spot after just one week of application of the topical composition. Daily application of the topical composition for four weeks as described in Example 2 stimulated hair growth and resulted in hair growth at the bald spot, which was sufficient to cover up the subject's bald spot.

### Example 8

In referring to FIG. 6, the subject, having braids, has noticeable bald spots between each set of braids, almost in a grid-like pattern. Daily application of the topical composition for four weeks as described in Example 2 stimulated hair growth and resulted in hair growth at the bald spots between the braids such that the bald spots were no longer noticeable.

### Example 9

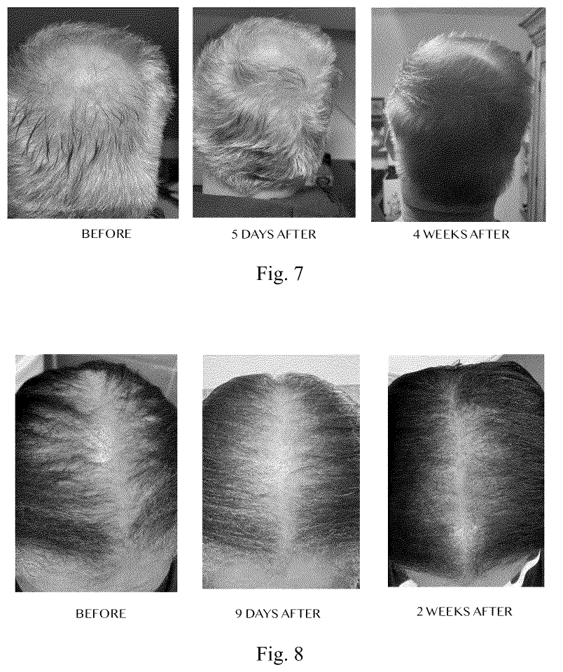

FIG. 7 shows a balding subject prior to treatment. The hair was noticeably thicker after just five days of application of the topical composition. Daily application of the topical composition for four weeks as described in Example 2 stimulated hair growth and resulted in hair growth that covered up the original balding area.

### Example 10

FIG. 8 shows the treatment course of a female subject exhibiting female pattern baldness. The bald spot began noticeably fill in with new hair growth after just nine days of application of the topical composition. Daily application of the topical composition for two weeks as described in Example 2 stimulated hair growth and resulted in hair growth to better cover the balding spot.

### Example 11

The topical composition may be used as a moisture sealant following application of other hair care products such as gel or creams. After the initial hair care treatment with the gel or cream, approximately 1 tablespoon (˜15 mL) of the topical composition is applied to the scalp with massaging for at least one minute. The topical composition is allowed to fully absorb and form a protecting layer on the hair follicle to seal in the moisture and is not rinsed or washed out following application.

### Example 12

The topical composition can also be used as a component in the hot oil treatment of one's hair. Between about 50 to about 100 mL of the topical composition is heated on the stovetop or in the microwave prior to application to the scalp and hair. The volume of composition needed depends on the size of the treated area as well as the amount of hair on the subject. The composition, after heating, should be just warm to the touch. An ideal temperature is between about 75° to about 130° F. although personal preference and heat tolerance will ultimately determine the subject's ideal temperature for the treatment. The heated topical composition is applied to the hair and scalp and left in for approximately 10 to 15 min to allow the warmed up oil to penetrate the scalp, roots, and hair follicles. After treatment, the topical composition is washed out.

### Example 13

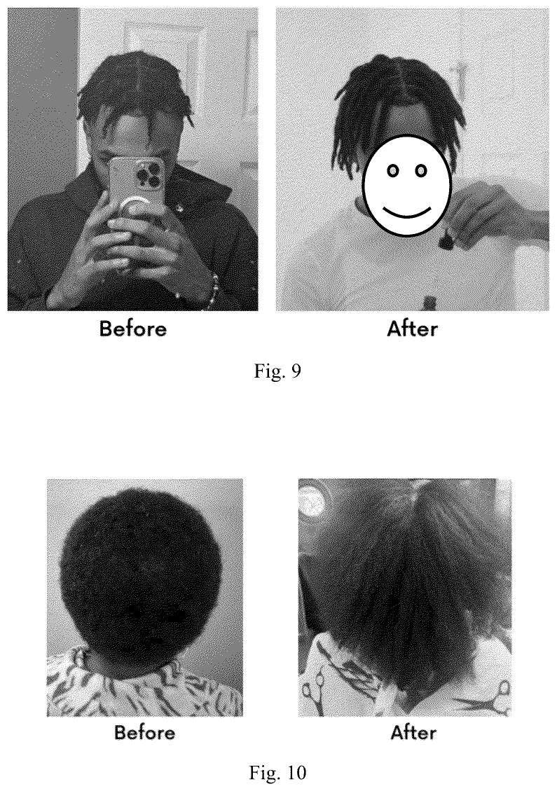

The topical composition maintains its effect in stimulating hair growth and increasing hair density and length with continual application as shown in FIGS. 9 and 10. FIG. 9 shows a male subject bearing dreadlocks but short hair length prior to treatment. After one year of application of the topical composition according to Example 2, 2-3 times weekly, to stimulate hair growth, the subject exhibited longer and thicker hair as shown in the right hand photograph of FIG. 9. FIG. 10 shows a female subject having short hair prior to treatment. Application of the topical composition according to Example 2, 2-3 times weekly, stimulated hair growth and the hair growth rate to provide the long tresses of hair as observed in the right hand photograph of FIG. 10.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A topical composition comprising an active component and an essential oil component, the active component comprising at least one oil selected from the group consisting of camellia oil, safflower oil, jojoba oil, vitamin E oil, coconut oil, rose hip oil, moringa oil, grapeseed oil, marula oil, Jamaican black castor oil, olive oil, avocado oil, neem oil, brahmi infused sesame oil, and bhringraj infused sesame oil, and combinations thereof; and
the essential oil component comprising at least one essential oil.

2. The topical composition of claim 1, wherein the at least one essential oil is selected from the group consisting of peppermint oil, rosemary oil, tea tree oil, lemongrass oil, elemi oil, lavender oil, basil oil, eucalyptus oil, geranium oil, juniper oil, palmrose oil, passion fruit oil, patchouli oil, rosewood oil, sage oil, thyme oil, and ylang ylang oil, and combinations thereof.

3. The topical composition of claim 1, wherein the composition comprises as the active component by volume of the composition (in v/v %):
about 5% to 15% camellia oil;
about 10% to about 20% safflower oil;
about 5% to about 15% Jojoba oil;
about 0.01% to about 5% vitamin E oil;
about 10% to about 25% coconut oil;
about 0.01% to about 10% rose hip oil;
about 0.01% to about 5% moringa oil;
about 5% to about 15% grapeseed oil;
about 0.01% to about 10% marula oil;
about 0.01% to about 10% Jamaican black castor oil;
about 0.01% to about 10% olive oil;
about 5% to 15% avocado oil;
about 0.01% to about 5% neem oil;
about 0.01% to about 5% brahmi infused sesame oil; and
about 0.01% to about 5% bhringraj infused sesame oil.

4. The composition of claim 1, wherein the at least one essential oil comprises by volume of the composition (in v/v %):
about 0.01% to about 0.5% peppermint oil;
about 0.01% to about 0.3% rosemary oil;
about 0.01% to about 0.2% tea tree oil;
about 0.01% to about 0.3% lemongrass oil;
about 0.1% to about 0.2% elemi oil;
about 0.01% to about 0.4% lavender oil;
about 0.01% to about 0.1% basil oil;
about 0.01% to about 0.1% eucalyptus oil;
about 0.01% to about 0.1% geranium oil;
about 0.01% to about 0.1% juniper oil;
about 0.01% to about 0.1% palmrose oil;
about 0.01% to about 0.1% passion fruit oil;
about 0.01% to about 0.1% patchouli oil;
about 0.01% to about 0.1% rosewood oil;
about 0.01% to about 0.1% sage oil;
about 0.01% to about 0.1% thyme oil; and
about 0.01% to about 0.1% ylang ylang oil.

5. The topical composition of claim 1, wherein the active component is at least 97% by volume of the composition.

6. The topical composition of claim 1, wherein the essential oil component is at most 3% by volume of the composition.

7. The topical composition of claim 5, wherein the active component is at least 98.5% by volume of the composition.

8. The topical composition of claim 6, wherein the essential oil component is at most 1.5% by volume of the composition.

9. The topical composition of claim 1, wherein a volume ratio of coconut oil to safflower oil ranges from 0.5:1 to 2.5:1.

10. The topical composition of claim 3, wherein the volume ratio of coconut oil to safflower oil is 1.33:1.

11. The topical composition of claim 1, wherein the topical composition does not contain any nut-based oils.

12. The topical composition of claim 5, wherein the topical composition does not contain silicones, sulfates, petroleums, mineral oil, parabens, phthalates, and formaldehyde.

13. The topical composition of claim 1, wherein the active component comprises (in v/v %):
about 5% to 15% camellia oil;
about 10% to about 20% safflower oil;
about 5% to about 15% Jojoba oil;
about 0.01% to about 5% vitamin E oil;
about 10% to about 25% coconut oil;
about 0.01% to about 10% rose hip oil;
about 0.01% to about 5% moringa oil;
about 5% to about 15% grapeseed oil;
about 0.01% to about 10% marula oil;
about 0.01% to about 10% Jamaican black castor oil;
about 0.01% to about 10% olive oil;
about 5% to 15% avocado oil;
about 0.01% to about 5% neem oil;
about 0.01% to about 5% brahmi infused sesame oil; and
about 0.01% to about 5% bhringraj infused sesame oil;
and the essential oil component comprises:
about 0.01% to about 0.5% peppermint oil;
about 0.01% to about 0.3% rosemary oil;
about 0.01% to about 0.2% tea tree oil;
about 0.01% to about 0.3% lemongrass oil;
about 0.1% to about 0.2% elemi oil;
about 0.01% to about 0.4% lavender oil;
about 0.01% to about 0.1% basil oil;
about 0.01% to about 0.1% eucalyptus oil;
about 0.01% to about 0.1% geranium oil;
about 0.01% to about 0.1% juniper oil;
about 0.01% to about 0.1% palmrose oil;
about 0.01% to about 0.1% passion fruit oil;
about 0.01% to about 0.1% patchouli oil;
about 0.01% to about 0.1% rosewood oil;
about 0.01% to about 0.1% sage oil;
about 0.01% to about 0.1% thyme oil; and
about 0.01% to about 0.1% ylang ylang oil.

14. A topical composition, comprising an active component, wherein the active component comprises:
8 fl. oz. camellia oil;
12 fl. oz. safflower oil;
6 fl. oz. Jojoba oil;
2 fl. oz. vitamin E oil;
16 fl. oz. coconut oil;
4 fl. oz. rose hip oil;
2 fl. oz. moringa oil;
8 fl. oz. grapeseed oil;
4 fl. oz. marula oil;
4 fl. oz. Jamaican black castor oil;
4 fl. oz. olive oil;
8 fl. oz. avocado oil;
2 fl. oz. neem oil;
2 fl. oz. brahmi infused sesame oil; and
2 fl. oz. bhringraj infused sesame oil;
and an essential oil component, wherein the essential oil component comprises:
1.25 tsp peppermint oil;
0.75 tsp rosemary oil;
0.5 tsp tea tree oil;
0.75 tsp lemongrass oil;
0.5 tsp elemi oil;
1 tsp lavender oil;
0.25 tsp basil oil;
0.25 tsp eucalyptus oil;
0.25 tsp geranium oil;
0.25 tsp juniper oil;
0.25 tsp palmrose oil;
0.25 tsp passion fruit oil;
0.25 tsp patchouli oil;
0.25 tsp rosewood oil;
0.25 tsp sage oil;
0.25 tsp thyme oil; and
0.25 tsp ylang ylang oil.

15. A method for stimulating hair follicles for hair regrowth, strengthening hair, thickening hair, moisturizing dry hair or scalp, or reducing dandruff or scalp irritation comprising applying a topical composition to a scalp, the composition comprising:
an active component comprising at least one oil selected from the group consisting of camellia oil, safflower oil, jojoba oil, vitamin E oil, coconut oil, rose hip oil, moringa oil, grapeseed oil, marula oil, Jamaican black castor oil, olive oil, avocado oil, neem oil, brahmi infused sesame oil, and bhringraj infused sesame oil, and combinations thereof; and
an essential oil component comprising at least one essential oil selected from the group consisting of peppermint oil, rosemary oil, tea tree oil, lemongrass oil, elemi oil, lavender oil, basil oil, eucalyptus oil, geranium oil, juniper oil, palmrose oil, passion fruit oil, patchouli oil, rosewood oil, sage oil, thyme oil, and ylang ylang oil, and combinations thereof.

16. The method of claim 15, comprising applying between about 0.5 mL to about 30 mL of the composition to the scalp.

17. The method of claim 15, comprising applying between about 1 mL to about 15 mL of the composition to the scalp.

18. The method of claim 15, wherein applying occurs once daily.

19. The method of claim 15, comprising heating the composition to a temperature between about 100° to about 175° F. before applying the composition to the scalp.

20. The method of claim 19, wherein stimulating hair follicles for hair regrowth or thickening hair comprises increasing hair density from about 10 hairs per square inch of a scalp to about 300 hairs per square inch on the scalp.
